SEQUENCE LISTING (8 sequences)

SEQ ID NO: 1 - DNA, 1139 bp, Homo sapiens
  CDS (5)...(979)
caggatgcagggccgcgtggcagggagctgcgctcctctgggcctgctcctggtctgtcttcatctcccaggcctctttgcccggagcatcggtgttgtggaggagaaagtttcccaaaacttcgggaccaacttgcctcagctcggacaaccttcctccactggcccctctaactctgaacatccgcagcccgctctggaccctaggtctaatgacttggcaagggttcctctgaagctcagcgtgcctccatcagatggcttcccacctgcaggaggttctgcagtgcagaggtggcctccatcgtgggggctgcctgccatggattcctggccccctgaggatccttggcagatgatggctgctgcggctgaggaccgcctgggggaagcgctgcctgaagaactctcttacctctccagtgctgcggccctcgctccgggcagtggccctttgcctggggagtcttctcccgatgccacaggcctctcacctgaggcttcactcctccaccaggactcggagtccagacgactgccccgttctaattcactgggagccgggggaaaaatcctttcccaacgccctccctggtctctcatccacagggttctgcctgatcacccctggggtaccctgaatcccagtgtgtcctggggaggtggaggccctgggactggttggggaacgaggcccatgccacaccctgagggaatctggggtatcaataatcaacccccaggtaccagctggggaaatattaatcggtatccaggaggcagctggggaaatattaatcggtatccaggaggcagctgggggaatattaatcggtatccaggaggcagctgggggaatattcatctatacccaggtatcaataacccatttcctcctggagttctccgccctcctggctcttcttggaacatcccagctggcttccctaatcctccaagccctaggttgcagtggggctagagcacgatagagggaaacccaacattgggagttagagtcctgctcccgccccttgctgtgtgggctcaatccaggccctgttaacatgtttccagcactatccccacttttcagtgcctcccctgctcatctccaataaaataaaagcacttatgga

SEQ ID NO: 2 - protein, 325 aa, Homo sapiens [sequence omitted]

SEQ ID NO: 3 - DNA, 975 bp, Artificial Sequence
  Degenerate sequence
atgcarggnmgngtngcnggnwsntgygcnccnytnggnytnytnytngtntgyytncayytnccnggnytnttygcnmgnwsnathggngtngtngargaraargtnwsncaraayttyggnacnaayytnccncarytnggncarccnwsnwsnacnggnccnwsnaaywsngarcayccncarccngcnytngayccnmgnwsnaaygayytngcnmgngtnccnytnaarytnwsngtnccnccnwsngayggnttyccnccngcnggnggnwsngcngtncarmgntggccnccnwsntggggnytnccngcnatggaywsntggccnccngargayccntggcaratgatggcngcngcngcngargaymgnytnggngargcnytnccngargarytnwsntayytnwsnwsngcngcngcnytngcnccnggnwsnggnccnytnccnggngarwsnwsnccngaygcnacnggnytnwsnccngargcnwsnytnytncaycargaywsngarwsnmgnmgnytnccnmgnwsnaaywsnytnggngcnggnggnaarathytnwsncarmgnccnccntggwsnytnathcaymgngtnytnccngaycayccntggggnacnytnaayccnwsngtnwsntggggnggnggnggnccnggnacnggntggggnacnmgnccnatgccncayccngarggnathtggggnathaayaaycarccnccnggnacnwsntggggnaayathaaymgntayccnggnggnwsntggggnaayathaaymgntayccnggnggnwsntggggnaayathaaymgntayccnggnggnwsntggggnaayathcayytntayccnggnathaayaayccnttyccnccnggngtnytnmgnccnccnggnwsnwsntggaayathccngcnggnttyccnaayccnccnwsnccnmgnytncartggggn

SEQ ID NO: 4 - DNA, 24 bp, Artificial Sequence
  Oligonucleotide primer ZC28251
tgctcctggtctgtcttcatctcc

SEQ ID NO: 5 - DNA, 23 bp, Artificial Sequence
  Oligonucleotide primer ZC28252
cagtgaattagaacggggcagtc

SEQ ID NO: 6 - DNA, 32 bp, Artificial Sequence
  Oligonucleotide primer ZC 27355
tatattggccggccaccatgcagggccgcgtg

SEQ ID NO: 7 - DNA, 32 bp, Artificial Sequence
  Oligonucleotide primer ZC27356
gtatacggcgcgccctagccccactgcaacct

SEQ ID NO: 8 - DNA, 1257 bp, Homo sapiens
  CDS (19)...(996)
gaattcggctcgagcaggatgcagggccgcgtggcagggagctgcgctcctctgggcctgctcctggtctgtcttcatctcccaggcctctttgcccggagcatcggtgttgtggaggagaaagtttcccaaaacttcgggaccaacttgcctcagctcggacaaccttcctccactggcccctctaactctgaacatccgcagcccgctctggaccctaggtctaatgacttggcaagggttcctctgaagctcagcgtgcctccatcagatggcttcccacctgcaggaggttctgcagtgcagaggtggcctccatcgtgggggctgcctgccatggattcctggccccctgaggatccttggcagatgatggctgctgcggctgaggaccgcctgggggaagcgctgcctgaagaactctcttacctctccagtgctgcggccctcgctccgggcagtggccctttgcctggggagtcttctcccgatgccacaggcctctcacctgaggcttcactcctccaccaggactcggagtccagacgactgccccgttctaattcactgggagccgggggaaaaatcctttcccaacgccctccctggtctctcatccacagggttctgcctgatcacccctggggtaccctgaatcccagtgtgtcctggggaggtggaggccctgggactggttggggaacgaggcccatgccacaccctgagggaatctggggtatcaataatcaacccccaggtaccagctggggaaatattaatcggtatccaggaggcagctggggaaatattaatcggtatccaggaggcagctgggggaatattaatcggtatccaggaggcagctgggggaatattcatctatacccaggtatcaataacccatttcctcctggagttctccgccctcctggctcttcttggaacatcccagctggcttccctaatcctccaagccctaggttgcagtggggctagagcacgatagagggaaacccaacattgggagttagagtcctgctcccgccccttgctgtgtgggctcaatccaggccctgttaacatgtttccagcactatccccacttttcagtgcctcccctgctcatctccaataaaataaaagcacttatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagggcggccgc

(19) United States
(12) Patent Application Publication
(10) Pub. No.: US 2002/0042095 A1
(43) Pub. Date: Apr. 11, 2002

(54) Secreted protein, ZSIG89

(76) Inventors: David A. Adler, Bainbridge Island, WA (US); Paul O. Sheppard, Granite Falls, WA (US); Andrew J. Nelson, Shoreline, WA (US)

Correspondence Address: Robyn Adams, ZymoGenetics, Inc., 1201 Eastlake Avenue East, Seattle, WA, 98102, US

(21) Appl. No.: 09/741,711

(22) Filed: Dec. 19, 2000

Related U.S. Application Data
(60) Provisional application No. 60/172,972, filed on Dec. 21, 1999.

(51) Int. Cl. C12Q 1/68; C07H 21/04; C12P 21/02; C12N 5/06

(52) U.S. Cl. 435/69.1; 435/6; 435/70.21; 435/325; 536/23.5; 530/350; 530/388.1

(57) ABSTRACT

Novel secreted BAT-related polypeptides, polynucleotides encoding the polypeptides, antibodies and related compositions and methods are disclosed. The polypeptides may be used for detecting receptors, agonists and antagonists. The polypeptides, polynucleotides and antibodies may also be used in methods that modulate inflammation, infection, and immunity.

### REFERENCE TO RELATED APPLICATIONS

This application is related to Provisional Applications 60/172,972 filed on Dec. 21, 1999. Under 35 U.S.C. §119(e)(1), this application claims benefit of said Provisional Application.

### BACKGROUND OF THE INVENTION

The major histocompatibility complex (MHC) is a chromosomal region that controls the immunological response. Experimental analysis of this system has revealed the MHC to be multifunctional and play a role in virtually every aspect of the immune response. Located within the MHC are the highly polymorphic class I and class II genes that are responsible for coding proteins involved in the presentation of processed antigens to the T cell receptor. Thus, infected cells are killed and antibodies are raised to foreign antigens. Between class I and class II genes are the class III genes which consist of a number of unrelated genes with low polymorphism including genes encoding steroid, 21-hydroxylase, complement components, heat shock proteins, tumor necrosis factor, and a number of genes with unknown function. See Snoek, M. et al., Immunogentics 49: 468-470, 1999.

Highly conserved between human and mouse, the MHC contains many peptide presentation genes as well as other genes whose products relate to T cell function, such as peptidases, peptide transporters and cytokines. Population studies suggest that susceptibility to a number of autoimmune diseases is associated with certain MHC haplotypes. Many of these genetic associations may be related to polymorphisms in class I and class II molecules. In some cases this increased susceptibility may be due to the combinatorial effect of several gene products. Since MHC haplotypes specify allelic combinations of a number of genes linked within the MHC, it is possible that genes lying between the class I and class II gene families could contribute to disease pathology. See Banerji, J. et al., Proc. Natl. Acad. Sci. USA 87: 2374-2378, 1990.

Human Leukocyte Antigen B Associated Transcripts (BATs) map to the class III region of the human MHC region at chromosomal location 6p21.3. This protein family, contains proteins whose functions seem unrelated: BAT3 contains a peptide fragment that interacts with DAN, a candidate tumor suppresser (Ozaki, T. et al., DNA Cell Biol. 18: 503-512, 1999); BAT6 encodes a valyl-tRNA synthetase; and BAT1 and BAT5, which, are suspected to be involved in ankylosing spondylitis based on their proximity to the HLA-B antigen.

BAT2 and BAT3 genes encode large proline-rich proteins with repeated domains. Other proline-rich proteins include transcriptional regulatory proteins containing zinc finger motifs and proline- or glutamine-rich regions, the oncogene homolog, elk, collagens, elastin, and synapsin. Additionally, polyproline and polyglycine, and individual collagen chains are able to adopt a common helical structure, so it is possible that a similar conformation might be assumed by some on the proline- and/or glycine-rich regions in BAT2 and BAT3. See Banerji, supra.

The chromosomal location of MHC is 6p21. As this locus has a high density of immunologically related, novel genes, which map to this region are important for medical research and treatment of diseases relating to the immune response.

### DESCRIPTION OF THE INVENTION

Within one aspect, the invention provides an isolated polypeptide comprising residues 27 to 325 of SEQ ID NO:2. Within an embodiment, the polypeptide comprises residues 1 to 325 of SEQ ID NO:2.

Within another aspect, the invention provides an isolated polypeptide selected from the group consisting of: a polypeptide comprising residues 1 to 26 of SEQ ID NO:2; a polypeptide comprising residues 27 to 325 of SEQ ID NO:2; and a polypeptide comprising residues 1 to 325 of SEQ ID NO:2.

Within another aspect, is provided, an isolated polynucleotide encoding a polypeptide wherein the polypeptide comprises residues 27 to 325 of SEQ ID NO:2. Within an embodiment, the polypeptide molecule comprises residues 1 to 325 of SEQ ID NO:2. Within another embodiment, the invention provides an isolated polynucleotide, wherein the isolated polypeptide has an amino acid sequence that is at least 90% identical to the polypeptide sequence as shown in SEQ ID NO:2. Within another embodiment, any difference between the isolated polypeptide and the polypeptide as shown in SEQ ID NO:2 is due to conservative amino acid substitution.

Within another aspect, the invention provides an isolated polynucleotide encoding a polypeptide molecule wherein the polypeptide is selected from the group consisting of: a polypeptide comprising residues 1 to 26 of SEQ ID NO:2; a polypeptide comprising residues 27 to 325 of SEQ ID NO:2; and a polypeptide comprising residues 1 to 325 of SEQ ID NO:2. Within an embodiment, the invention provides an expression vector comprising the following operably linked elements: a) a transcription promoter; b) a DNA segment wherein the DNA segment is a polynucleotide encoding polypeptide comprising amino acid residues 27 to 325 of SEQ ID NO:2; and a transcription terminator. Within another embodiment, the DNA segment contains an affinity tag. Within another embodiment, is provided a cultured cell into which has been introduced the expression vector, wherein said cell expresses the polypeptide encoded by the DNA segment. Within another embodiment, the invention provides a method of producing a polypeptide comprising culturing the cell, whereby said cell expresses the polypeptide encoded by the DNA segment; and recovering the polypeptide. Within an embodiment, the invention provides the polypeptide produced.

Within another aspect, the invention provides a method of producing an antibody comprising the following steps in order: inoculating an animal with a polypeptide selected from the group consisting of: a polypeptide comprising residues 1 to 26 of SEQ ID NO:2; a polypeptide comprising residues 27 to 325 of SEQ ID NO:2; and a polypeptide comprising residues 1 to 325 of SEQ ID NO:2; wherein the polypeptide elicits an immune response in the animal to produce the antibody; and isolating the antibody from the animal. Within an embodiment, the antibody binds to a polypeptide comprising residues 1 to 325 of SEQ ID NO:2. Within another embodiment, the antibody is a monoclonal antibody.

Within another aspect is provided an antibody which specifically binds to a polypeptide comprising residues 1 to 325 of SEQ ID NO:2. Within an embodiment, the antibody is a monoclonal antibody.

Within another aspect, the invention provides a method of producing an antibody comprising the following steps in order: inoculating an animal with an epitope bearing portion of a polypeptide wherein the epitope bearing portion is selected from the group consisting of: a polypeptide consisting of residues 1 to 8 of SEQ ID NO:2; a polypeptide consisting of residues 30 to 44 of SEQ ID NO:2; a polypeptide consisting of residues 47 to 77 of SEQ ID NO:2; a polypeptide consisting of residues 81 to 94 of SEQ ID NO:2; a polypeptide consisting of residues 97 to 102 of SEQ ID NO:2; a polypeptide consisting of residues 108 to 117 of SEQ ID NO:2; a polypeptide consisting of residues 97 to 117 of SEQ ID NO:2; a polypeptide consisting of residues 108 to 136 of SEQ ID NO:2; a polypeptide consisting of residues 122 to 136 of SEQ ID NO:2; a polypeptide consisting of residues 146 to 197 of SEQ ID NO:2; a polypeptide consisting of residues 203 to 209 of SEQ ID NO:2; a polypeptide consisting of residues 203 to 238 of SEQ ID NO:2; a polypeptide consisting of residues 218 to 238 of SEQ ID NO:2; a polypeptide consisting of residues 242 to 250 of SEQ ID NO:2 a polypeptide consisting of residues; 242 to 271 of SEQ ID NO:2; a polypeptide consisting of residues 252 to 271 of SEQ ID NO:2; a polypeptide consisting of residues 274 to 283 of SEQ ID NO:2; a polypeptide consisting of residues 252 to 283 of SEQ ID NO:2; a polypeptide consisting of residues 295 to 308 of SEQ ID NO:2; a polypeptide consisting of residues 311 to 324 of SEQ ID NO:2; wherein the polypeptide elicits an immune response in the animal to produce the antibody; and isolating the antibody from the animal. Within an embodiment, the antibody binds to a polypeptide comprising residues 1 to 325 of SEQ ID NO:2. Within another embodiment, the antibody is a monoclonal antibody.

Within another aspect, the invention provides a method of producing an antibody comprising the following steps in order: inoculating an animal with a polypeptide selected from the group consisting of: a polypeptide comprising residues 1 to 26 of SEQ ID NO:2; a polypeptide comprising residues 27 to 325 of SEQ ID NO:2; and a polypeptide comprising residues 1 to 325 of SEQ ID NO:2, wherein the polypeptide elicits an immune response in the animal to produce the antibody; and isolating the antibody from the animal. Within an embodiment the invention provides the antibody produced by this method. Within another embodiment, the antibody produced specifically binds to a residues 1 to 325 of SEQ ID NO:2.

Within another aspect the invention provides an epitope-bearing portion of the polypeptide as shown in SEQ ID NO:2, wherein the epitope bearing portion comprises 30 contiguous amino acids. Within an embodiment, is provided a method of producing an antibody comprising the following steps in order: inoculating an animal with an epitope-bearing portion that comprises 30 contiguous amino acids as shown in SEQ ID NO:2; wherein the polypeptide elicits an immune response in the animal; producing the antibody in the animal; and isolating the antibody from the animal, wherein the antibody binds to the epitope-bearing portion. Within an embodiment the invention provides the antibody produced by this method. Within another embodiment, the antibody specifically binds to residues 1 to 325 of SEQ ID NO:2.

Within another aspect the invention provides an antibody that binds to a polypeptide comprising residues 1 to 325 of SEQ ID NO:2, wherein the antibody is a monoclonal antibody.

Within another aspect is provided a method of detecting the presence of ZSIG89 gene expression in a genetic sample, comprising: obtaining the genetic sample; incubating the genetic sample with a polynucleotide probe or primer, wherein the polynucleotide probe or primer comprises a portion of the polynucleotide consisting of nucleotide 76 to nucleotide 979 of SEQ ID NO:1, under conditions wherein the polynucleotide will hybridize to a complementary polynucleotide sequence; producing a reaction product; and detecting the formation of hybrids of the polynucleotide probe or primer and the genetic sample in the reaction product, wherein the presence of the hybrids indicates the presence of ZSIG89 gene expression in the genetic sample.

Within another aspect the invention provides a method of detecting the presence of ZSIG89 gene expression in a biological sample, comprising: obtaining the biological sample; contacting the biological sample with an antibody or antibody fragment under conditions wherein the contacting allows the binding of the antibody or antibody fragment to the biological sample, wherein the antibody or antibody fragment specifically binds with a polypeptide consisting of amino acid residues 27 to 325 of SEQ ID NO:2; and detecting the presence bound antibody or antibody fragment, wherein the presence of the bound antibody or antibody fragment indicates the presence of ZSIG89 gene expression.

These and other aspects of the invention will become evident upon reference to the following detailed description of the invention.

Prior to setting forth the invention in detail, it may be helpful to the understanding thereof to define the following terms:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985) (SEQ ID NO:7), substance P, Flag™ peptide (Hopp et al., Biotechnology 6:1204-1210, 1988), streptavidin binding peptide, maltose binding protein (Guan et al., Gene 67:21-30, 1987), cellulose binding protein, thioredoxin, ubiquitin, T7 polymerase, or other antigenic epitope or binding domain. See, in general, Ford et al., Protein Expression and Purification 2: 95-107, 1991. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “corresponding to”, when applied to positions of amino acid residues in sequences, means corresponding positions in a plurality of sequences when the sequences are optimally aligned.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “ortholog” or “species homolog”, denotes a polypeptide or protein obtained from one species that is the functional counterpart of a polypeptide or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-domain or multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel 1139 bp cDNA sequence (SEQ ID NO:1) and corresponding polypeptide sequence (SEQ ID NO:2) which has been designated, ZSIG89. SEQ ID NO:8 shows the cDNA and additional 5′ and 3′ untranslated DNA sequence. Novel ZSIG89 ligand-encoding polynucleotides and polypeptides of the present invention were identified by the discovery of a novel cDNA from a lung tumor (squamous cell carcinoma). Sequence analysis of a deduced amino acid sequence indicates the presence of a signal sequence (residues 1 to 26 of SEQ ID NO:2), and a proline-leucine-serine-tryptophan rich domain (PLSW domain) of residues 27 to 325 of SEQ ID NO:2. ZSIG89 maps to human chromosome 6 within band p21.3. These features indicate that the ligand encoded by the DNA sequence of SEQ ID NO:1 is a member of the BAT protein family. Those skilled in the art will recognize that these domain boundaries are approximate, and are based on alignments with known proteins and predictions of protein folding.

Analysis of the tissue distribution of ZSIG89 can be performed by the Northern blotting technique using Human Multiple Tissue and Master Dot Blots. Such blots are commercially available (Clontech, Palo Alto, Calif.) and can be probed by methods known to one skilled in the art. Also see, for example, Wu W. et al., Methods in Gene Biotechnology, CRC Press LLC, 1997. Additionally, portions of the polynucleotides of the present invention can be identified by querying sequence databases and identifying the tissues from, which the sequences are derived. Portions of the polynucleotides of the present invention have been identified in squamous cell carcinoma lung tumor cDNA libraries. Similarly, tissue expression of the ZSIG89 molecules of the present invention can be identified by polymerase chain reaction analysis of various tissues and cell lines. This analysis shows that ZSIG89 cDNA has been identified in fetal liver, fetal skin, spinal cord, trachea, lung tumor, rectal tumor, and genomic DNA.

The present invention provides polynucleotide molecules, including DNA and RNA molecules, that encode the ZSIG89 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the ZSIG89 polypeptide of SEQ ID NO:2. Those skilled in the art will recognize that the degenerate sequence of SEQ ID NO:3 also provides all RNA sequences encoding SEQ ID NO:2 by substituting U for T. Thus, ZSIG89 polypeptide-encoding polynucleotides comprising nucleotide 1 to nucleotide 1139 of SEQ ID NO:1, nucleotide 1 to nucleotide 1257 of SEQ ID NO:7, and their RNA equivalents are contemplated by the present invention. Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCAGCCGCGGCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

The present invention further provides polynucleotide molecules, including DNA and RNA molecules, encoding ZSIG89 proteins. The polynucleotides of the present invention include the sense strand; the anti-sense strand; and the DNA as double-stranded, having both the sense and anti-sense strand annealed together by their respective hydrogen bonds. Representative DNA sequences encoding ZSIG89 proteins are set forth in SEQ ID NOs:1 and 3. DNA sequences encoding other ZSIG89 proteins can be readily generated by those of ordinary skill in the art based on the genetic code.

One of ordinary skill in the art will also appreciate that different species can exhibit “preferential codon usage.” Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed in SEQ ID NO:3 serve as templates for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NO:1, or a sequence complementary thereto under stringent conditions. Polynucleotide hybridization is well known in the art and widely used for many applications, see for example, Sambrook et al., Molecular Cloning: A Laboratory Manual, Second Edition, Cold Spring Harbor, N.Y., 1989; Ausubel et al., eds., Current Protocols in Molecular Biology, John Wiley and Sons, Inc., NY, 1987; Berger and Kimmel, eds., Guide to Molecular Cloning Techniques, Methods in Enzymology, volume 152, 1987 and Wetmur, Crit. Rev. Biochem. Mol. Biol. 26:227-59, 1990. Polynucleotide hybridization exploits the ability of single stranded complementary sequences to form a double helix hybrid. Such hybrids include DNA-DNA, RNA-RNA and DNA-RNA.

As an illustration, a nucleic acid molecule encoding a variant ZSIG89 polypeptide can be hybridized with a nucleic acid molecule having the nucleotide sequence of SEQ ID NO:1 (or its complement) at 65° C. overnight in ExpressHyb™ Hybridization Solution (CLONTECH Laboratories, Inc., Palo Alto, Calif.). One of skill in the art can devise variations of these hybridization conditions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant ZSIG89 polypeptide hybridize with a nucleic acid molecule having the nucleotide sequences of SEQ ID NO:1 (or its complement) under stringent washing conditions, in which the wash stringency is equivalent to 0.1×-2×SSC with 0.1% SDS at 55-65° C., including 0.1×SSC with 0.1% SDS at 55° C., or 2×SSC with 0.1% SDS at 65° C. One of skill in the art can readily devise equivalent conditions, for example, by substituting SSPE for SSC in the wash solution.

The present invention also contemplates ZSIG89 variant nucleic acid molecules that can be identified using two criteria: a determination of the similarity between the encoded polypeptides with the amino acid sequences of SEQ ID NO:2 (as described below), and a hybridization assay, as described above. Such ZSIG89 variants include nucleic acid molecules that hybridize with a nucleic acid molecule having the nucleotide sequence of SEQ ID NO:1 (or its complement) under stringent washing conditions, in which the wash stringency is equivalent to 0.1×-2×SSC with 0.1% SDS at 55-65° C., and (2) that encode a polypeptide having at least 80%, preferably 90%, more preferably, 95% or greater than 95% sequence identity to the amino acid sequence of SEQ ID NO:2. Alternatively, ZSIG89 variants can be characterized as nucleic acid molecules (1) that hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., and (2) that encode a polypeptide having at least 80%, preferably 90%, more preferably 95% or greater than 95% sequence identity to the amino acid sequence of SEQ ID NO:2.

The highly conserved amino acids in the PLSW domain of ZSIG89 can be used as a tool to identify new family members. For instance, reverse transcription-polymerase chain reaction (RT-PCR) can be used to amplify sequences encoding the conserved domain from RNA obtained from a variety of tissue sources or cell lines. In particular, highly degenerate primers designed from the ZSIG89 sequences are useful for this purpose.

As previously noted, the isolated polynucleotides of the present invention include DNA and RNA. Methods for preparing DNA and RNA are well known in the art. In general, RNA is isolated from a tissue or cell that produces large amounts of ZSIG89 RNA. Such tissues and cells can be identified by Northern blotting (Thomas, Proc. Natl. Acad. Sci. USA 77:5201, 1980), and may include tumorous lung.

Total RNA can be prepared using guanidine isothiocyante extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)+RNA using known methods. In the alternative, genomic DNA can be isolated. Polynucleotides encoding ZSIG89 polypeptides are then identified and isolated by, for example, hybridization or PCR.

A full-length clone encoding ZSIG89 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to ZSIG89 or other specific binding partners.

The invention also provides isolated and purified ZSIG89 polynucleotide probes. Such polynucleotide probes can be RNA or DNA. DNA can be either cDNA or genomic DNA. Polynucleotide probes are single or double-stranded DNA or RNA, generally synthetic oligonucleotides, but may be generated from cloned cDNA or genomic sequences and will generally comprise at least 16 nucleotides, more often from 17 nucleotides to 25 or more nucleotides, sometimes 40 to 60 nucleotides, and in some instances a substantial portion, domain or even the entire ZSIG89 gene or cDNA. The synthetic oligonucleotides of the present invention have at least 80% identity to a representative ZSIG89 DNA sequence (SEQ ID NOs: 1 or 3) or their complements. The invention also provides oligonucleotide probes or primers comprising at least 14 contiguous nucleotides of a polynucleotide of SEQ ID NOs: 1 or 3 or a sequence complementary to SEQ ID NOs: 1 or 3.

Preferred regions from which to construct probes include the 5′ and/or 3′ coding sequences, ligand binding regions, and signal sequences, and the like. Techniques for developing polynucleotide probes and hybridization techniques are known in the art, see for example, Ausubel et al., eds., Current Protocols in Molecular Biology, John Wiley and Sons, Inc., NY, 1991. For use as probes, the molecules can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art. Such probes can also be used in hybridizations to detect the presence or quantify the amount of ZSIG89 gene or mRNA transcript in a sample. ZSIG89 polynucleotide probes could be used to hybridize to DNA or RNA targets for diagnostic purposes, using such techniques such as fluorescent in situ hybridization (FISH) or immunohistochemistry. Polynucleotide probes can be used to identify genes encoding ZSIG89-like proteins. For example, ZSIG89 polynucleotides can be used as primers and/or templates in PCR reactions to identify other novel members of the BAT protein family. Such probes can also be used to screen libraries for related sequences encoding novel similar proteins. Such screening would be carried out under conditions of low stringency which would allow identification of sequences which are substantially homologous, but not requiring complete homology to the probe sequence. Such methods and conditions are well known in the art, see, for example, Sambrook et al., Molecular Cloning: A Laboratory Manual, Second Edition, Cold Spring Harbor, N.Y., 1989. Such low stringency conditions could include hybridization temperatures less than 42° C., formamide concentrations of less than 50% and moderate to low concentrations of salt. Libraries may be made of genomic DNA or cDNA. Polynucleotide probes are also useful for Southern, Northern, or slot blots, colony and plaque hybridization and in situ hybridization. Mixtures of different ZSIG89 polynucleotide probes can be prepared which would increase sensitivity or the detection of low copy number targets, in screening systems.

In addition, such polynucleotide probes could be used to hybridize to counterpart sequences on individual chromosomes. Chromosomal identification and/or mapping of the ZSIG89 gene could provide useful information about gene function and disease association. Many mapping techniques are available to one skilled in the art, for example, mapping somatic cell hybrids, and fluorescence in situ hybridization (FISH). A preferred method is radiation hybrid mapping. Radiation hybrid mapping is a somatic cell genetic technique developed for constructing high-resolution, contiguous maps of mammalian chromosomes (Cox et al., Science 250:245-50, 1990). Partial or full knowledge of a gene's sequence allows the designing of PCR primers suitable for use with chromosomal radiation hybrid mapping panels. Commercially available radiation hybrid mapping panels which cover the entire human genome, such as the Stanford G3 RH Panel and the GeneBridge 4 RH Panel (Research Genetics, Inc., Huntsville, Ala.), are available. These panels enable rapid, PCR based, chromosomal localizations and ordering of genes, sequence-tagged sites (STSs), and other non-polymorphic- and polymorphic markers within a region of interest. This includes establishing directly proportional physical distances between newly discovered genes of interest and previously mapped markers. The precise knowledge of a gene's position can be useful in a number of ways including: 1) determining if a sequence is part of an existing contig and obtaining additional surrounding genetic sequences in various forms such as YAC-, BAC- or cDNA clones, 2) providing a possible candidate gene for an inheritable disease which shows linkage to the same chromosomal region, and 3) for cross-referencing model organisms such as mouse which may be beneficial in helping to determine what function a particular gene might have.

ZSIG89 polynucleotide sequences disclosed herein can also be used as probes or primers to clone 5′ non-coding regions of a ZSIG89 gene. In view of the tissue observation for ZSIG89 in lung squamous cell carcinoma, this gene region is expected to provide for specific expression in tissues lining the respiratory and gastrointestinal tracts, including lung and oral tissues. Promoter elements from a ZSIG89 gene could thus be used to direct the tissue-specific expression of heterologous genes in, for example, transgenic animals or patients treated with gene therapy. Cloning of 5′ flanking sequences also facilitates production of ZSIG89 proteins by “gene activation” as disclosed in U.S. Pat. No. 5,641,670. Briefly, expression of an endogenous ZSIG89 gene in a cell is altered by introducing into the ZSIG89 locus a DNA construct comprising at least a targeting sequence, a regulatory sequence, an exon, and an unpaired splice donor site. The targeting sequence is a ZSIG89 5′ non-coding sequence that permits homologous recombination of the construct with the endogenous ZSIG89 locus, whereby the sequences within the construct become operably linked with the endogenous ZSIG89 coding sequence. In this way, an endogenous ZSIG89 promoter can be replaced or supplemented with other regulatory sequences to provide enhanced, tissue-specific, or otherwise regulated expression.

The polynucleotides of the present invention can also be synthesized using DNA synthesizers. Currently the method of choice is the phosphoramidite method. If chemically synthesized double stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. See Glick and Pasternak, Molecular Biotechnology, Principles and Applications of Recombinant DNA, (ASM Press, Washington, D.C. 1994); Itakura et al., Annu. Rev. Biochem. 53: 323-356 (1984) and Climie et al., Proc. Natl. Acad. Sci. USA 87:633-7, 1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are ZSIG89 polypeptides from other mammalian species, including murine, porcine, ovine, bovine, canine, feline, equine, and other primate polypeptides. Orthologs of human ZSIG89 can be cloned using information and compositions provided by the present invention in combination with conventional cloning techniques. For example, a cDNA can be cloned using mRNA obtained from a tissue or cell type that expresses ZSIG89 as disclosed herein. Such tissue would include, for example, tissues lining the respiratory and gastrointestinal tracts, including lung and oral tissues. Suitable sources of mRNA can be identified by probing Northern blots with probes designed from the sequences disclosed herein. A library is then prepared from mRNA of a positive tissue or cell line. A ZSIG89-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human ZSIG89 sequences disclosed herein. Within an additional method, the cDNA library can be used to transform or transfect host cells, and expression of the cDNA of interest can be detected with an antibody to ZSIG89 polypeptide. Similar techniques can also be applied to the isolation of genomic clones.

Those skilled in the art will recognize that the sequences disclosed in SEQ ID NO:1 represents a single allele of human ZSIG89 and that allelic variation and alternative splicing are expected to occur. Allelic variants of this sequence can be cloned by probing cDNA or genomic libraries from different individuals according to standard procedures. Allelic variants of the DNA sequences shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the ZSIG89 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art. As stated earlier, polynucleotides of SEQ ID NO:1 and SEQ ID NO:3 are alternatively spliced variants of the same gene.

The present invention also provides isolated ZSIG89 polypeptides that are substantially similar to the polypeptides of SEQ ID NO:2 and their orthologs. Such polypeptides will be about 80%, or 85% more preferably be at least 90% identical, and more preferably 95% or more identical to SEQ ID NO:2 and their orthologs. Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of indentical matches}{\begin{matrix}[ length of the longer sequence plus the \\ \begin{matrix}number of gaps introduced into the longer \\ sequence in order to align the two sequences ]\end{matrix}\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant ZSIG89. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from four to six.

The present invention includes nucleic acid molecules that encode a polypeptide having one or more conservative amino acid changes, compared with the amino acid sequences of SEQ ID NO:2. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. As used herein, the language “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Conservative amino acid changes in an ZSIG89 gene can be introduced by substituting nucleotides for the nucleotides recited in SEQ ID NO:1. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)). The ability of such variants to modulate cell-cell interactions, infectivity, and inflammation can be determined using a standard method, such as the assay described herein. Alternatively, a variant ZSIG89 polypeptide can be identified by the ability to specifically bind anti-ZSIG89 antibodies.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of receptor-ligand interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related BAT related molecules.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed ZSIG89 DNA and polypeptide sequences can be generated through DNA shuffling, as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., receptor binding activity) can be recovered from the host cells and rapidly sequenced using modem equipment. These methods allow the rapid determination of the importance of individual amino acid residues in a polypeptide of interest, and can be applied to polypeptides of unknown structure.

Regardless of the particular nucleotide sequence of a variant ZSIG89 gene, the gene encodes a polypeptide that is characterized by its receptor binding activity, including but not limited to tumorigensis, as well as modulation of bacterial or viral adhesion to the epithelium, or by the ability to bind specifically to an anti-ZSIG89 antibody. More specifically, variant ZSIG89 genes encode polypeptides which exhibit at least 50%, and preferably, greater than 70, 80, or 90%, of the activity of polypeptide encoded by the human ZSIG89 gene described herein.

Variant ZSIG89 polypeptides or substantially homologous ZSIG89 polypeptides are characterized as having one or more amino acid substitutions, deletions or additions. These changes are preferably of a minor nature, that is conservative amino acid substitutions and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from 299 to 2000 amino acid residues that comprise a sequence that is at least 85%, preferably at least 90%, and more preferably 95% or more identical to the corresponding region of SEQ ID NO:2. Polypeptides comprising affinity tags can further comprise a proteolytic cleavage site between the ZSIG89 polypeptide and the affinity tag. Preferred such sites include thrombin cleavage sites and factor Xa cleavage sites.

For any ZSIG89 polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise ZSIG89 variants based upon the nucleotide and amino acid sequences described herein. Accordingly, the present invention includes a computer-readable medium encoded with a data structure that provides at least one of the following sequences: SEQ ID NO:1, SEQ ID NO:2, and SEQ ID NO:3. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

The present invention further provides a variety of other polypeptide fusions and related multimeric proteins comprising one or more polypeptide fusions. For example, the PLSW domain can be prepared as a fusion to a dimerizing protein, as disclosed in U.S. Pat. Nos. 5,155,027 and 5,567,584. Preferred dimerizing proteins in this regard include other proline-rich domains, or polypeptides comprising other members of the BAT family of proteins, such as, for example, BAT2 and BAT3. These polypeptide domain fusions, can be expressed in genetically engineered cells to produce a variety of multimeric BAT related analogs.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. For example, part or all of a domain(s) conferring a biological function may be swapped between ZSIG89 of the present invention with the functionally equivalent domain(s) from another family member, such as BAT2 and BAT3. Such domains include, but are not limited to, conserved motifs such as the PLSW domain. Such fusion proteins would be expected to have a biological functional profile that is the same or similar to polypeptides of the present invention or other known BAT family of proteins (e.g. BAT2 and BAT3), depending on the fusion constructed. Moreover, such fusion proteins may exhibit other properties as disclosed herein.

Moreover, using methods described in the art, polypeptide fusions, or hybrid ZSIG89 proteins, are constructed using regions or domains of the inventive ZSIG89 in combination with those of other BAT related molecules. (e.g. BAT2 and BAT3), or heterologous proteins (Sambrook et al., ibid., Altschul et al., ibid., Picard, Cur. Opin. Biology, 5:511-5, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Auxiliary domains can be fused to ZSIG89 polypeptides to target them to specific cells, tissues, or macromolecules (e.g., epithelial tissues of the respiratory tract, oral cavity and gut epithelium). For example, a protease domain could be targeted to a predetermined cell type (tumorous lung) by fusing it to a the PLSW domain (residues 26 to 325 of SEQ ID NO:2), or a portion thereof which has been shown to bind the cognate receptor for ZSIG89. In this way, polypeptides, polypeptide fragments and proteins can be targeted for therapeutic or diagnostic purposes. Such PLSW domain, or portions thereof can be fused to two or more moieties, such as an affinity tag for purification and a targeting domains. Polypeptide fusions can also comprise one or more cleavage sites, particularly between domains. See, Tuan et al., Connective Tissue Research 34:1-9, 1996.

Polypeptide fusions of the present invention will generally contain not more than about 1,500 amino acid residues, preferably not more than about 1,200 residues, more preferably not more than about 1,000 residues, and will in many cases be considerably smaller. For example, residues of ZSIG89 polypeptide can be fused to E. coli β-galactosidase (1,021 residues; see Casadaban et al., J. Bacteriol. 143:971-980, 1980), a 10-residue spacer, and a 4-residue factor Xa cleavage site. In a second example, residues of ZSIG89 polypeptide can be fused to maltose binding protein (approximately 370 residues), a 4-residue cleavage site, and a 6-residue polyhistidine tag.

The invention also provides soluble ZSIG89, used to form fusion or chimeric proteins with human Ig, as His-tagged proteins, or FLAG™-tagged proteins. One such construct is comprises residues 27 to 325 of SEQ ID NO:2, fused to human Ig. ZSIG89 or ZSIG89-Ig chimeric proteins are used, for example, to identify the ZSIG89 receptor, including the natural receptor, as well as agonists and antagonists of the natural receptor. Using labeled soluble ZSIG89, cells expressing the receptor are identified by fluorescence immunocytometry or immunohistochemistry. The soluble fusion proteins or soluble Ig fusion protein is useful in studying the distribution of the receptor on tissues or specific cell lineages, and to provide insight into receptor/ligand biology.

In an alternative approach, a ZSIG89 receptor-binding region can be expressed as a chimera with immunoglobulin heavy chain constant regions, typically an F_{c} fragment, which contains two constant region domains and a hinge region, but lacks the variable region. Such fusions are typically secreted as multimeric molecules, wherein the Fc portions are disulfide bonded to each other and two ligand polypeptides are arrayed in proximity to each other. Fusions of this type can be used to affinity purify the cognate receptor from solution, as an in vitro assay tool, to block signals in vitro by specifically titrating out receptor, and as agonists in vivo by administering them to simulate ligand binding. To purify receptor, a ZSIG89-Ig fusion protein (chimera) is added to a sample containing the receptor under conditions that facilitate receptor-ligand binding (typically near-physiological temperature, pH, and ionic strength). The chimera-receptor complex is then separated by the mixture using protein A, which is immobilized on a solid support (e.g., insoluble resin beads). The receptor is then eluted using conventional chemical techniques, such as with a salt or pH gradient. In the alternative, the chimera itself can be bound to a solid support, with binding and elution carried out as above. For use in assays, the chimeras are bound to a support via the F_{c} region and used in an ELISA format.

To direct the export of a ZSIG89 polypeptide from the host cell, the ZSIG89 DNA may be linked to a second DNA segment encoding a secretory peptide, such as a t-PA secretory peptide or a ZSIG89 secretory peptide. To facilitate purification of the secreted polypeptide, a C-terminal extension, such as a poly-histidine tag, substance P, Flag peptide (Hopp et al., Bio/Technology 6:1204-1210, 1988; available from Eastman Kodak Co., New Haven, Conn.), maltose binding protein, or another polypeptide or protein for which an antibody or other specific binding agent is available, can be fused to the ZSIG89 polypeptide.

The present invention also includes “functional fragments” of ZSIG89 polypeptides and nucleic acid molecules encoding such functional fragments. Routine deletion analyses of nucleic acid molecules can be performed to obtain functional fragments of a nucleic acid molecule that encodes an ZSIG89 polypeptide. As an illustration, DNA molecules having the nucleotide sequence of SEQ ID NO:1 can be digested with Bal31 nuclease to obtain a series of nested deletions. The fragments are then inserted into expression vectors in proper reading frame, and the expressed polypeptides are isolated and tested for cell-cell interactions, or for the ability to bind anti-ZSIG89 antibodies. One alternative to exonuclease digestion is to use oligonucleotide-directed mutagenesis to introduce deletions or stop codons to specify production of a desired fragment. Alternatively, particular fragments of an ZSIG89 gene can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of a ZSIG89 gene that has amino acid changes, compared with the amino acid sequence of SEQ ID NO:2. A variant ZSIG89 gene can be identified on the basis of structure by determining the level of identity with nucleotide and amino acid sequences of SEQ ID NOs:1 and 2, as discussed above. An alternative approach to identifying a variant gene on the basis of structure is to determine whether a nucleic acid molecule encoding a potential variant ZSIG89 gene can hybridize to a nucleic acid molecule having the nucleotide sequence of SEQ ID NO:1, as discussed above.

Using the methods discussed herein, one of ordinary skill in the art can identify and/or prepare a variety of polypeptide fragments or variants of SEQ ID NO:2 or that retain the receptor-binding, or intracellular signaling activity of the wild-type ZSIG89 protein. Such polypeptides may include additional amino acids from the PLSW and secretory domains, including amino acids responsible for intracellular signaling; fusion domains; affinity tags; and the like.

Within the polypeptides of the present invention are polypeptides that comprise an epitope-bearing portion of a protein as shown in SEQ ID NO:2. An “epitope” is a region of a protein to which an antibody can bind. See, for example, Geysen et al., Proc. Natl. Acad. Sci. USA 81:3998-4002, 1984. Epitopes can be linear or conformational, the latter being composed of discontinuous regions of the protein that form an epitope upon folding of the protein. Linear epitopes are generally at least 6 amino acid residues in length. Relatively short synthetic peptides that mimic part of a protein sequence are routinely capable of eliciting an antiserum that reacts with the partially mimicked protein. See, Sutcliffe et al., Science 219:660-666, 1983. Antibodies that recognize short, linear epitopes are particularly useful in analytic and diagnostic applications that employ denatured protein, such as Western blotting (Tobin, Proc. Natl. Acad. Sci. USA 76:4350-4356, 1979), or in the analysis of fixed cells or tissue samples. Antibodies to linear epitopes are also useful for detecting fragments of ZSIG89, such as might occur in body fluids or cell culture media.

Antigenic, epitope-bearing polypeptides of the present invention are useful for raising antibodies, including monoclonal antibodies, that specifically bind to a ZSIG89 protein. Antigenic, epitope-bearing polypeptides contain a sequence of at least six, preferably at least nine, more preferably from 15 to about 30 contiguous amino acid residues of a ZSIG89 protein (e.g., SEQ ID NO:2). Polypeptides comprising a larger portion of a ZSIG89 protein, i.e. from 30 to 50 residues up to the entire sequence, are included. It is preferred that the amino acid sequence of the epitope-bearing polypeptide is selected to provide substantial solubility in aqueous solvents, that is the sequence includes relatively hydrophilic residues, and hydrophobic residues are substantially avoided. Preferred such regions include the PLSW domain, or the secretory domain of ZSIG89 and fragments thereof. Specific, preferred polypeptides in this regard include those comprising residues 1 to 26 of SEQ ID NO:2; residues 27 to 325 of SEQ ID NO:2; and residues 1 to 325 of SEQ ID NO:2.

The present invention also provides polypeptide fragments or peptides comprising an epitope-bearing portion of an ZSIG89 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides contain at least four to ten amino acids, preferably at least ten to fifteen amino acids, more preferably 15 to 30 amino acids of SEQ ID NO:2. Such epitope-bearing peptides and polypeptides can be produced by fragmenting an ZSIG89 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5and pages 9.4.1-9.4.11 (John Wiley & Sons 1997).

ZSIG89 polypeptides can also be used to prepare antibodies that specifically bind to ZSIG89 epitopes, peptides or polypeptides. The ZSIG89 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a ZSIG89 polypeptide (e.g., SEQ ID NO:2). Polypeptides comprising a larger portion of a ZSIG89 polypeptide, i.e., from 30 to 10 residues up to the entire length of the amino acid sequence are included. Antigens or immunogenic epitopes can also include attached tags, adjuvants and carriers, as described herein. Suitable antigens include the ZSIG89 polypeptides encoded by SEQ ID NO:2 from amino acid number 1 to amino acid number 325, or a contiguous 9 to 325 amino acid fragment thereof.

As an illustration, potential antigenic sites in ZSIG89 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

Suitable antigens from this analysis include residue 1 to residue 8 of SEQ ID NO:2; residue 30 to 44 of SEQ ID NO:2; residue 47 to residue 77 of SEQ ID NO:2; residue 81 to residue 94 of SEQ ID NO:2; residue 97 to residue 102 of SEQ ID NO:2; residue 108 to residue 117 of SEQ ID NO:2; residue 97 to residue 117 of SEQ ID NO:2 ; residue 108 to residue 136 of SEQ ID NO:2; residue 122 to residue 136 of SEQ ID NO:2; residue 146 to residue 197 of SEQ ID NO:2; residue 203 to residue 209 of SEQ ID NO:2 ; residue 203 to residue 238 of SEQ ID NO:2; residue 218 to residue 238 of SEQ ID NO:2; residue 242 to residue 250 of SEQ ID NO:2; residue 242 to residue 271 of SEQ ID NO:2; residue 252 to residue 271 of SEQ ID NO:2; residue 274 to residue 283 of SEQ ID NO:2; residue 252 to residue 283 of SEQ ID NO:2; residue 295 to residue 308 of SEQ ID NO:2 ; residue 311 to residue 324 of SEQ ID NO:2; and residue 295 to residue 324 of SEQ ID NO:2; or a portion thereof which contains a 4 to amino acid segment. Hydrophilic peptides, such as those predicted by one of skill in the art from a hydrophobicity plot are also immonogenic. ZSIG89 hydrophilic peptides include peptides comprising amino acid sequences selected from the group consisting of: residues 34 to 43 of SEQ ID NO:2; residues 45 to 74 of SEQ ID NO:2; residues 84 to 89 of SEQ ID NO:2; residues 93 to 102 of SEQ ID NO:2; residues 84 to 102 of SEQ ID NO:2; residues 108 to 117 of SEQ ID NO:2; residues 93 to 117 of SEQ ID NO:2; residue 125 to residue 130 of SEQ ID NO:2; residue 150 to residue 158 of SEQ ID NO:2; residue 167 to residue 181 of SEQ ID NO:2; residue 150 to residue 181 of SEQ ID NO:2; residue 191 to residue 198 of SEQ ID NO:2; residue 204 to residue 212 of SEQ ID NO:2; residue 191 to residue 212 of SEQ ID NO:2; residue 221 to residue 236 of SEQ ID NO:2; residue 238 to residue 283 of SEQ ID NO:2; residue 291 to residue 298 of SEQ ID NO:2; residue 301 to residue 309 of SEQ ID NO:2; and residue 291 to residue 309 of SEQ ID NO:2; or a portion thereof which contains a 4 to 10 amino acid segment. Additionally, antigens can be generated to portions of the polypeptide which are likely to be on the surface of the folded protein. These antigens include: residue 34 to residue 40 of SEQ ID NO:2; residue 50 to residue 71 of SEQ ID NO:2; residue 34 to residue 71 of SEQ ID NO:2; residue 109 to residue 116 of SEQ ID NO:2; residue 151 to residue 159 of SEQ ID NO:2; residue 171 to residue 184 SEQ ID NO:2; residue 171 to residue 196 of SEQ ID NO:2; residue 191 to residue 196 of SEQ ID NO:2; residue 228 to residue 234 of SEQ ID NO:2; residue 242 to residue 247 of SEQ ID NO:2; residue 228 to residue 247 of SEQ ID NO:2; and residue 315 to residue 322 of SEQ ID NO:2 ; or a portion thereof which contains a 4 to 10 amino acid segment.

Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a ZSIG89 polypeptide or a fragment thereof. The immunogenicity of a ZSIG89 polypeptide may be increased through the use of an adjuvant, such as alum (aluminum hydroxide) or Freund's complete or incomplete adjuvant. Polypeptides useful for immunization also include fusion polypeptides, such as fusions of ZSIG89 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization. Similarly, ZSIG89 polypeptides, themselves are immunostimulatory and can be used as an adjuvant for vaccination.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to ZSIG89 protein or peptide, and selection of antibody display libraries in phage or similar vectors (for instance, through use of immobilized or labeled ZSIG89 protein or peptide). Genes encoding polypeptides having potential ZSIG89 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from CLONTECH Laboratories, Inc., (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the ZSIG89 sequences disclosed herein to identify proteins which bind to ZSIG89. These “binding proteins” which interact with ZSIG89 polypeptides can be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as ZSIG89 “antagonists” to block ZSIG89 binding and signal transduction in vitro and in vivo. These anti-ZSIG89 binding proteins would be useful for modulating, for example, infection, inflammation, immunologic recognition, tumor formation, and cell-cell interactions in general.

As used herein, the term “binding proteins” additionally includes antibodies to ZSIG89 polypeptides, the cognate receptor of ZSIG89 polypeptides, proteins useful for purification of ZSIG89 polypeptides, and proteins associated with the PLSW domain (residues 27 to 325 of SEQ ID NO:2).

Antibodies are determined to be specifically binding if they exhibit a threshold level of binding activity (to a ZSIG89 polypeptide, peptide or epitope) of at least 10-fold greater than the binding affinity to a control (non-ZSIG89) polypeptide. The binding affinity of an antibody can be readily determined by one of ordinary skill in the art, for example, by Scatchard analysis (Scatchard, G., Ann. NY Acad. Sci. 51: 660-672, 1949).

A variety of assays known to those skilled in the art can be utilized to detect antibodies which specifically bind to ZSIG89 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant ZSIG89 protein or polypeptide.

Antibodies to ZSIG89 may be used for immunohistochemical tagging cells that express ZSIG89; for isolating ZSIG89 by affinity purification; for diagnostic assays for determining circulating levels of ZSIG89 polypeptides; for detecting or quantitating soluble ZSIG89 as marker of underlying pathology or disease; in analytical methods employing FACS; for screening expression libraries; for generating anti-idiotypic antibodies; and as neutralizing antibodies or as antagonists to block ZSIG89 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to ZSIG89 or fragments thereof may be used in vitro to detect denatured ZSIG89 or fragments thereof in assays, for example, Western Blots or other assays known in the art.

The soluble ZSIG89 is useful in studying the distribution of receptors on tissues or specific cell lineages, and to provide insight into receptor/ligand biology. Using labeled ZSIG89, cells expressing the receptor are identified by fluorescence immunocytometry or immunocytochemistry.

Antibodies can be made to soluble ZSIG89 polypeptides which are His or FLAG™ tagged. Alternatively, such polypeptides form a fusion protein with Human Ig. In particular, antiserum containing polypeptide antibodies to His-tagged, or FLAG™-tagged soluble ZSIG89 can be used in analysis of tissue distribution of ZSIG89 by immunohistochemistry on human or primate tissue. These soluble ZSIG89 polypeptides can also be used to immunize mice in order to produce monoclonal antibodies to a soluble human ZSIG89 polypeptide. Monoclonal antibodies to a soluble human ZSIG89 polypeptide can also be used to mimic ligand/receptor coupling, resulting in activation or inactivation of the ligand/receptor pair. For instance, it has been demonstrated that cross-linking anti-soluble CD40 monoclonal antibodies provides a stimulatory signal to B cells that have been sub-optimally activated with anti-IgM or LPS, and results in proliferation and immunoglobulin production. These same monoclonal antibodies act as antagonists when used in solution by blocking activation of the receptor. Monoclonal antibodies to ZSIG89 can be used to determine the distribution, regulation and biological interaction of the ZSIG89/ZSIG89-receptor pair on specific cell lineages identified by tissue distribution studies.

Soluble ZSIG89 or antibodies to ZSIG89 can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, ZSIG89 polypeptides or anti-ZSIG89 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/ anticomplementary pair.

In another embodiment, polypeptide-toxin fusion proteins or antibody-toxin fusion proteins can be used for targeted cell or tissue inhibition or ablation (for instance, to treat cancer or inflammation in cells or tissues). Alternatively, a fusion protein including only the PLSW domain may be suitable for directing a detectable molecule, a cytotoxic molecule or a complementary molecule to a cell or tissue type of interest. Similarly, the corresponding receptor to ZSIG89 can be conjugated to a detectable or cytotoxic molecule and provide a generic targeting vehicle for cell/tissue-specific delivery of generic anti-complementary-detectable/ cytotoxic molecule conjugates.

In another embodiment, ZSIG89-cytokine fusion proteins or antibody-cytokine fusion proteins can be used for enhancing in vivo killing of target tissues (for example, in the lung), if the ZSIG89 polypeptide or anti-ZSIG89 antibody targets hyperproliferative tissues from these organs. (See, generally, Homick et al., Blood 89:4437-47, 1997). They described fusion proteins that enable targeting of a cytokine to a desired site of action, thereby providing an elevated local concentration of cytokine. Suitable ZSIG89 polypeptides or anti-ZSIG89 antibodies target an undesirable cell or tissue (i.e., infection, a tumor or a leukemia), and the fused cytokine mediates improved target cell lysis by effector cells. Suitable cytokines for this purpose include interleukin 2 and granulocyte-macrophage colony-stimulating factor (GM-CSF), for instance.

ZSIG89 polynucleotides and/or polypeptides may be useful for regulating the maturation of ZSIG89 receptor-bearing cells, such as T cells, B cells, lymphocytes, peripheral blood mononuclear cells, polymorphonuclear leukocytes, fibroblasts and hematopoietic cells. ZSIG89 polypeptides will also find use in mediating metabolic or physiological processes in vivo. The effects of a compound on proliferation and differentiation can be measured in vitro using cultured cells. Bioassays and ELISAs are available to measure cellular response to ZSIG89, in particular are those which measure changes in cytokine production as a measure of cellular response (see for example, Current Protocols in Immunology ed. John E. Coligan et al., NIH, 1996). Assays to measure other cellular responses, including antibody isotype, monocyte activation, tumorigenesis, and bacterial and viral clearance are known in the art.

The ZSIG89 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a ZSIG89 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a ZSIG89 polypeptide into the secretory pathway of a host cell, a secretory signal sequence (also known as a leader sequence, prepro sequence or pre sequence) can be provided in the expression vector. The secretory signal sequence may be derived from ZSIG89 (i.e., residues 1 to 26 of SEQ ID NO:2) or another secreted protein (e.g., BAT3, or t-PA) or synthesized de novo. The secretory signal sequence is operably linked to the ZSIG89 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

The PLSW domain of ZSIG89 can be substituted by a heterologous sequence providing a different PLSW domain. In this case, the fusion product can be secreted, and the secretory domain of ZSIG89 can direct the new PLSW domain to a specific tissue described above. This substituted PLSW domain can be chosen from the PLSW domain represented by a member of the BAT protein family. Similarly, the secretory domain of ZSIG89 protein can be substituted by a heterlogous sequence providing a different secretory domain. Again, the fusion product can be secreted and the substituted secretory domain can target the PLSW domain of ZSIG89 to a specific tissue. The substituted secretory domain can be chosen from the secretory domains of the BAT protein family.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins, such as CD4, CD8, Class I MHC, or placental alkaline phosphatase, may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant ZSIG89 baculovirus utilizes a transposon-based system described by Luckow (Luckow, V. A, et al., J Virol 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the ZSIG89 polypeptide into a baculovirus genome maintained in E. coli as a large plasmid called a “bacmid.” The pFastBac1™ transfer vector utilizes the AcNPV polyhedrin promoter to drive the expression of the gene of interest, in this case ZSIG89. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol Chem 270:1543-9, 1995. In such transfer vector constructs, a short or long version of the basic protein promoter can be used. Moreover, transfer vectors can be constructed which replace the native ZSIG89 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native ZSIG89 secretory signal sequence. In addition, transfer vectors can include an in-frame fusion with DNA encoding an epitope tag at the C- or N-terminus of the expressed ZSIG89 polypeptide, for example, a Glu-Glu epitope tag (Grussenmeyer, T. et al., Proc. Natl. Acad. Sci. 82:7952-4, 1985). Using a technique known in the art, a transfer vector containing ZSIG89 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses ZSIG89 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. #5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the ZSIG89 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillennondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,716,808, 5,736,383, 5,854,039, and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a ZSIG89 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 10 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for ZSIG89 amino acid residues.

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant ZSIG89 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., ibid. Maltose binding protein fusions are purified on an amylose column according to methods known in the art.

The polypeptides of the present invention can be isolated by a combination of procedures including, but not limited to, anion and cation exchange chromatography, size exclusion, and affinity chromatography. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp. 529-39). Within additional embodiments of the invention, a fusion of the polypeptide of interest and an affinity tag (e.g., maltose-binding protein, an immunoglobulin domain) may be constructed to facilitate purification.

ZSIG89 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Using methods known in the art, ZSIG89 proteins can be prepared as monomers or multimers; glycosylated or non-glycosylated; pegylated or non-pegylated; and may or may not include an initial methionine amino acid residue.

The activity of ZSIG89 polypeptides can be measured using a variety of assays that measure, for example, cell-cell interactions, inflammation, anti-infective capabilities, bacterial adherence and other biological functions associated with BAT protein family members or with BAT interactions, such as, differentiation, and proliferation for example. Assays measuring cell-cell interactions, inflammation, and infection and adherence are well known in the art.

Proteins, including alternatively spliced peptides, of the present invention are useful for tumor suppression, tumor staging, immunologic recognition, and growth and differentiation either working in isolation, or in conjunction with other molecules (growth factors, cytokines, etc.) in epithelial of the respiratory tract, and intestinal tract. Alternative splicing of ZSIG89 may cell-type specific and confer activity to specific tissues.

Another assay of interest measures or detects changes in proliferation, differentiation, and development. Additionally, the effects of a ZSIG89 polypeptides on cell-cell interactions of fibroblasts, epithelial cells, tumor cells and cells of the respiratory tract in particular, would be of interest to measure. Yet other assays examines changes in, infection, and intracellular signaling.

The activity of molecules of the present invention can be measured using a variety of assays that, for example, measure neogenesis or hyperplasia (i.e., proliferation) of tissues of the lung. Additional activities likely associated with the polypeptides of the present invention include proliferation of epithelial cells, fibroblasts, and lymphoid cells directly or indirectly through other growth factors; action as a chemotaxic factor for endothelial cells, fibroblasts and/or phagocytic cells; osteogenic factor; and factor for expanding mesenchymal stem cell and precursor populations.

The ZSIG89 polypeptides of the present invention can be used to study epithelial proliferation or differentiation in lung, as well as in the oral cavity, intestinal tract, and mucous membranes in general. Such methods of the present invention generally comprise incubating cells derived from these tissues in the presence and absence of ZSIG89 polypeptide, monoclonal antibody, agonist or antagonist thereof and observing changes in cell proliferation or differentiation. Cell lines from these tissues are commercially available from, for example, American Type Culture Collection (Manasas, Va.).

Proliferation can be measured using cultured lung cells or in vivo by administering molecules of the claimed invention to an appropriate animal model. Generally, proliferative effects are observed as an increase in cell number and therefore, may include inhibition of apoptosis, as well as mitogenesis. Cultured cells include uterine fibroblasts, lung tumors, and melanomas from primary cultures. Established cell lines are easily identifiable by one skilled in the art and are available from ATCC (Manasas, Va.). Assays measuring cell proliferation are well known in the art. For example, assays measuring proliferation include such assays as chemosensitivity to neutral red dye (Cavanaugh et al., Investigational New Drugs 8:347-354, 1990), incorporation of radiolabelled nucleotides (Cook et al., Analytical Biochem. 179:1-7, 1989), incorporation of 5-bromo-2′-deoxyuridine (BrdU) in the DNA of proliferating cells (Porstmann et al., J. Immunol. Methods 82:169-179, 1985), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:4827-4833, 1988).

To determine if ZSIG89 is a chemotractant in vivo, ZSIG89 can be given by intradermal or intraperitoneal injection. Characterization of the accumulated leukocytes at the site of injection can be determined using lineage specific cell surface markers and fluorescence immunocytometry or by immunohistochemistry (Jose, J. Exp. Med. 179:881-87, 1994). Release of specific leukocyte cell populations from bone marrow into peripheral blood can also be measured after ZSIG89 injection.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products and receptors and receptor-like complementary molecules. The stage of a cell population's differentiation is monitored by identification of markers present in the cell population. For example, myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention are useful for studies to isolate mesenchymal stem cells and lung fibroblast progenitor cells, both in vivo and ex vivo.

There is evidence to suggest that factors that stimulate specific cell types down a pathway towards terminal differentiation or dedifferentiation affect the entire cell population originating from a common precursor or stem cell. Thus, ZSIG89 polypeptides may stimulate inhibition or proliferation of endocrine and exocrine cells of the tissues lining the respiratory and gastrointestinal tracts, including lung and oral tissues.

Assays measuring differentiation include, for example, measuring cell-surface markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989).

Proteins, including alternatively spliced peptides, and fragments, of the present invention are useful for studying cell-cell interactions, immune recognition, growth control, tumor suppression, bacterial adherence, and inflammation. ZSIG89 molecules, variants, and fragments can be applied in isolation, or in conjunction with other molecules (growth factors, cytokines, etc.) in tissues lining the respiratory and gastrointestinal tracts, including lung and oral tissues.

Proteins of the present invention are useful for delivery of therapeutic agents such as, but not limited to, proteases, radionuclides, chemotherapy agents, and small molecules. Effects of these therapeutic agents can be measured in vitro using cultured cells, ex vivo on tissue slices, or in vivo by administering molecules of the claimed invention to the appropriate animal model. An alternative in vivo approach for assaying proteins of the present invention involves viral delivery systems. Exemplary viruses for this purpose include adenovirus, herpesvirus, lentivirus, vaccinia virus and adeno-associated virus (AAV). Adenovirus, a double-stranded DNA virus, is currently the best studied gene transfer vector for delivery of heterologous nucleic acid (for a review, see T. C. Becker et al., Meth. Cell Biol. 43:161-89, 1994; and J. T. Douglas and D. T. Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Garnier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

As a ligand, the activity of ZSIG89 polypeptide or a peptide to which ZSIG89 binds, can be measured by a silicon-based biosensor microphysiometer which measures the extracellular acidification rate or proton excretion associated with receptor binding interactions and subsequent physiologic cellular responses. An exemplary device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including ZSIG89 polypeptide, its agonists, and antagonists. ZSIG89-responsive eukaryotic cells comprise cells into which a polynucleotide for a receptor for ZSIG89 has been transfected creating a cell that is responsive to activation of ZSIG89; or cells naturally responsive to activation of ZSIG89. Differences, measured by a change in the response of cells exposed to ZSIG89 activation, relative to a control not exposed to ZSIG89 activation, are a direct measurement of ZSIG89-mediated cellular responses. Moreover, such ZSIG89-mediated responses can be assayed under a variety of stimuli. The present invention provides a method of identifying agonists and antagonists of ZSIG89 protein, comprising providing cells responsive to activation of ZSIG89 polypeptide, culturing a first portion of the cells in the absence of a test compound, culturing a second portion of the cells in the presence of a test compound, and detecting a change in a cellular response of the second portion of the cells as compared to the first portion of the cells. The change in cellular response is shown as a measurable change in extracellular acidification rate. Moreover, culturing a third portion of the cells in the presence of ZSIG89 polypeptide and the absence of a test compound provides a positive control for the ZSIG89-responsive cells, and a control to compare the agonist activity of a test compound with that of the ZSIG89 polypeptide. Antagonists of ZSIG89 can be identified by exposing the cells to ZSIG89 protein in the presence and absence of the test compound, whereby a reduction in ZSIG89-stimulated activity is indicative of antagonist activity in the test compound.

Similarly, the microphysiometer, can be used to rapidly identify cells, tissues, or cell lines which activate a ZSIG89-stimulated pathway. Such tissues and cell lines can be used to identify receptors, antagonists and agonists of ZSIG89 polypeptide as described above. Using similar methods, cells expressing ZSIG89 can be used to identify cells which stimulate or block a ZSIG89-signaling pathway.

ZSIG89, its agonists (including the native PLSW domain), and antagonists have enormous potential in both in vitro and in vivo applications. Compounds identified as ZSIG89 agonists and antagonists are useful for studying cell-cell interactions, tumor proliferation and suppression, infection, and inflammation in vitro and in vivo For example, ZSIG89 and agonist compounds are useful as components of defined cell culture media, and may be used alone or in combination with cytokines and hormones to replace serum that is commonly used in cell culture. Agonists are thus useful in specifically promoting the growth and/or development of cells of the myeloid and lymphoid lineages in culture. Additionally, ZSIG89 polypeptides and ZSIG89 agonists, including small molecules are useful as a research reagent, such as for the expansion, differentiation, proliferation, and/or cell-cell interactions of tissues lining the respiratory and gastrointestinal tracts, including lung and oral tissues. ZSIG89 polypeptides are added to tissue culture media for these cell types.

Compounds identified as ZSIG89 agonists are useful for modifying the proliferation and development of target cells in vitro and in vivo. For example, agonist compounds are useful alone or in combination with other cytokines and hormones as components of defined cell culture media. Agonists are thus useful in specifically mediating the growth and/or development of ZSIG89 receptor-bearing T lymphocytes or other ZSIG89 receptor-bearing cells in culture. Agonists and antagonists may also prove useful in the study of effector functions of T lymphocytes, in particular T lymphocyte activation and differentiation. Antagonists are useful as research reagents for characterizing ligand-receptor interaction.

The failure of tumor cell-tumor cell adhesion is believed to be a contributing factor in tumor metastases. See, for example, Zetter, Cancer Biology, 4: 219-29, 1993. Metastases, in turn, are generally associated with poor prognosis for cancer treatment. The metastatic process involves a variety of cellular events, including angiogenesis, tumor cell invasion of the vascular or lymphatic circulation, tumor cell arrest at a secondary site; tumor cell passage across the vessel wall into the parenchymal tissue, and tumor cell proliferation at the secondary site. Thus, both positive and negative regulation of adhesion are necessary for metastasis. That is, tumor cells must break away from the primary tumor mass, travel in circulation and adhere to cellular and/or extracellular matrix elements at a secondary site. Molecules capable of modulating cell-cell and cell-matrix adhesion are therefore sought for the study, diagnosis, prevention and/or treatment of metastases. Similar to BAT3, ZSIG89 polynucleotides and polypeptides may be involved in tumor and metastasis suppression.

As a member of the MHC class III proteins, ZSIG89 polypeptides are likely to be involved in the immune response to infection. ZSIG89 polypeptides, agonists, and antagonists can be used to treat microbial infections. Such infections include bacterial, yeast, and viral infections. Anti-microbial activity of proteins is evaluated by techniques that are known in the art. For example, anti-microbial activity can be assayed by evaluating the sensitivity of microbial cell cultures to test agents and by evaluating the protective effect of test agents on infected mice. See, for example, Musiek et al., Antimicrob. Agents Chemothr. 3:40, 1973. Antiviral activity can also be assessed by protection of mammalian cell cultures. Known techniques for evaluating anti-microbial activity include, for example, Barsum et al., Eur. Respir. J. 8:709-714, 1995; Sandovsky-Losica et al., J. Med. Vet. Mycol (England) 28:279-287, 1990; Mehentee et al., J. Gen. Microbiol (England) 135(:2181-2188, 1989; and Segal and Savage, J. Med. Vet. Mycol. 24:477-479, 1986. Assays specific for anti-viral activity include, for example, those described by Daher et al., J. Virol. 60:1068-1074, 1986. Similarly, assays measuring HIV-1 viral burden on cells can be used.

By nature of its proline-rich component ZSIG89 is similar to another group of proteins, the Proline-Rich Glycoproteins (PRGs). One protein in this group is Proline-Rich Glycoprotein (PRG), a basic protein especially rich in proline, glycine, and glutamic acid, which is found in high abundance only in parotid saliva. PRG has been demonstrated to be involved in lubrication. Additionally, it binds to bacteria by protein-protein interactions. See Amano, A. et al., Infection and Immunity 66:2072-2077, 1998. A proline- and glycine-rich repeat of PRG can bind Porphryomonas gingivalis. Thus PRG interferes with adherence, and infection, of this organism in the oral cavity.

In light of the presence of ZSIG89 in lung, and trachea, polypeptides and polynucleotides of the present invention can be useful in modulating bacterial adherence in these tissues. In one embodiment, bacterial fimbriae and flagella can be inhibited from adhering to lung tissue, thus inhibiting acute and chronic infection.

The invention also provides antagonists, which either bind to ZSIG89 polypeptides or, alternatively, to a receptor to which ZSIG89 polypeptides bind, thereby inhibiting or eliminating the function of ZSIG89. Such ZSIG89 antagonists would include antibodies; polypeptides which bind either to the ZSIG89 polypeptide or to its receptor; natural or synthetic analogs of ZSIG89 ligands which retain the ability to bind the receptor but do not result in either ligand or receptor signaling. Such analogs could be peptides or peptide-like compounds. Natural or synthetic small molecules which bind to ZSIG89 polypeptides and prevent signaling are also contemplated as antagonists. As such, ZSIG89 antagonists would be useful as therapeutics for treating certain disorders where blocking signal from either a ZSIG89 receptor or ligand would be beneficial. Antagonists are useful as research reagents for characterizing ligand-receptor interaction.

ZSIG89 polypeptides may be used within diagnostic systems to detect the presence of ligand polypeptides. Antibodies or other agents that specifically bind to ZSIG89 may also be used to detect the presence of circulating receptor or ligand polypeptides. Such detection methods are well known in the art and include, for example, enzyme-linked immunosorbent assay (ELISA) and radioimmunoassay. Immunohistochemically labeled ZSIG89 antibodies can be used to detect ZSIG89 receptor and/or ligands in tissue samples. ZSIG89 levels can also be monitored by such methods as RT-PCR, where ZSIG89 mRNA can be detected and quantified. The information derived from such detection methods would provide insight into the significance of ZSIG89 polypeptides in various diseases, and as such would serve as diagnostic tools for diseases for which altered levels of ZSIG89 are significant. Altered levels of ZSIG89 receptor polypeptides may be indicative of pathological conditions including cancer, autoimmune disorders, bone disorders, inflammation and immunodeficiencies.

Antagonists are also useful as research reagents for characterizing sites of interactions between members of complement/anti-complement pairs as well as sites of cell-cell interactions. Inhibitors of ZSIG89 activity (ZSIG89 antagonists) include anti-ZSIG89 antibodies and soluble ZSIG89 polypeptides (such as in SEQ ID NO:2), as well as other peptidic and non-peptidic agents (including ribozymes).

ZSIG89 can also be used to identify inhibitors (antagonists) of its activity. Test compounds are added to the assays disclosed herein to identify compounds that inhibit the activity of ZSIG89. In addition to those assays disclosed herein, samples can be tested for inhibition of ZSIG89 activity within a variety of assays designed to measure receptor/ligand binding or the stimulation/inhibition of ZSIG89-dependent cellular responses. For example, ZSIG89-responsive cell lines can be transfected with a reporter gene construct that is responsive to a ZSIG89-stimulated cellular pathway. Reporter gene constructs of this type are known in the art, and will generally comprise a DNA response element operably linked to a gene encoding an assayable protein, such as luciferase, or a metabolite, such as cyclic AMP. DNA response elements can include, but are not limited to, cyclic AMP response elements (CRE), hormone response elements (HRE), insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8): 1087-94; 1990. Hormone response elements are reviewed in Beato, Cell 56:335-44; 1989. Candidate compounds, solutions, mixtures or extracts are tested for the ability to inhibit the activity of ZSIG89 on the target cells, as evidenced by a decrease in ZSIG89 stimulation of reporter gene expression. Assays of this type will detect compounds that directly block ZSIG89 binding to a cell-surface protein, i.e., receptor, or the anti-complementary member of a complementary/anti-complementary pair, as well as compounds that block processes in the cellular pathway subsequent to complement/anti-complement binding. In the alternative, compounds or other samples can be tested for direct blocking of ZSIG89 binding to a receptor using ZSIG89 tagged with a detectable label (e.g., 1251, biotin, horseradish peroxidase, FITC, and the like). Within assays of this type, the ability of a test sample to inhibit the binding of labeled ZSIG89 to the receptor is indicative of inhibitory activity, which can be confirmed through secondary assays.

Also, ZSIG89 polypeptides, agonists or antagonists thereof may be therapeutically useful for promoting wound healing, for example, in tissues lining the respiratory and gastrointestinal tracts, including lung and oral tissues. To verify the presence of this capability in ZSIG89 polypeptides, agonists or antagonists of the present invention, such ZSIG89 polypeptides, agonists or antagonists are evaluated with respect to their ability to facilitate wound healing according to procedures known in the art. If desired, ZSIG89 polypeptide performance in this regard can be compared to growth factors, such as EGF, NGF, TGF-α, TGF-β, insulin, IGF-I, IGF-II, fibroblast growth factor (FGF) and the like. In addition, ZSIG89 polypeptides or agonists or antagonists thereof may be evaluated in combination with one or more growth factors to identify synergistic effects.

A ZSIG89 receptor-binding polypeptide can also be used for purification of receptor. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing receptors are passed through the column one or more times to allow receptors to bind to the ligand polypeptide. The receptor is then eluted using changes in salt concentration, chaotropic agents (guanidine HCl), or pH to disrupt ligand-receptor binding.

An assay system that uses a ligand-binding receptor (or an antibody, one member of a complementary/anti-complementary pair or other cell-surface binding protein) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complementary/anti-complementary pair is present in the sample, it will bind to the immobilized ligand, antibody or member, respectively, causing a change in the refractive index of the medium, which is detected as a change in surface plasmon resonance of the gold film. This system allows the determination of on- and off-rates, from which binding affinity can be calculated, and assessment of.

Receptor binding ligand polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

Molecules of the present invention can be used to identify and isolate receptors, or members of complement/anti-complement pairs involved in cell-cell interactions. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified.

The molecules of the present invention will be useful in modulating abnormal cell growth, inflammation, infection, proliferation and differentiation. The polypeptides, nucleic acid and/or antibodies of the present invention can be used in treatment of disorders associated with infection, tumor growth, immunodeficiency, and auto-immunity. The molecules of the present invention can be used to modulate infection, cell adhesion, and signaling or to treat or prevent development of pathological conditions in such diverse tissue as tissues lining the respiratory and gastrointestinal tracts, including lung and oral tissues. In particular, certain diseases may be amenable to such diagnosis, treatment or prevention. These diseases include, but are not limited to, immunodeficiency, autoimmunity, Insulin Dependent Diabetes Melitis, pneumonia, pneumocystis, Cystic Fibrosis, asthma, emphysema, allergies, cancer and infection. The molecules of the present invention can be used to modulate inhibition, inflammation and proliferation of tissues in the tissues lining the respiratory and gastrointestinal tracts, including lung and oral tissues, as well as epithelial tissues, in general.

Polynucleotides encoding ZSIG89 polypeptides are useful within gene therapy applications where it is desired to increase or inhibit ZSIG89 activity. If a mammal has a mutated or absent ZSIG89 gene, the ZSIG89 gene can be introduced into the cells of the mammal. In one embodiment, a gene encoding a ZSIG89 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a ZSIG89 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Feigner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Similarly, the ZSIG89 polynucleotides (SEQ ID NO:1 or SEQ ID NO:3) can be used to target specific tissues such as tissues lining the respiratory and gastrointestinal tracts, including lung and oral tissues. It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Various techniques, including antisense and ribozyme methodologies, can be used to inhibit ZSIG89 gene transcription and translation, such as to inhibit cell proliferation in vivo. Polynucleotides that are complementary to a segment of a ZSIG89-encoding polynucleotide (e.g., a polynucleotide as set forth in SEQ ID NOs: 1 or 3) are designed to bind to ZSIG89-encoding mRNA and to inhibit translation of such mRNA. Such antisense polynucleotides are used to inhibit expression of ZSIG89 polypeptide-encoding genes in cell culture or in a subject.

Mice engineered to express the ZSIG89 gene, referred to as “transgenic mice,” and mice that exhibit a complete absence of ZSIG89 gene function, referred to as “knockout mice,” may also be generated (Snouwaert et al., Science 257:1083, 1992), may also be generated (Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express ZSIG89 , either ubiquitously or under a tissue-specific or tissue-restricted promoter can be used to ask whether over-expression causes a phenotype. For example, over-expression of a wild-type ZSIG89 polypeptide, polypeptide fragment or a mutant thereof may alter normal cellular processes, resulting in a phenotype that identifies a tissue in which ZSIG89 expression is functionally relevant and may indicate a therapeutic target for the ZSIG89, its agonists or antagonists. For example, a transgenic mouse to engineer is one that over-expresses the mature ZSIG89 polypeptide (approximately amino acids 27 to 352 of SEQ ID NO:2), or the full-length ZSIG89 polypeptide, (residues 1 to 325 of SEQ ID NO:2). Moreover, such over-expression may result in a phenotype that shows similarity with human diseases. Similarly, knockout ZSIG89 mice can be used to determine where ZSIG89 is absolutely required in vivo. The phenotype of knockout mice is predictive of the in vivo effects of that a ZSIG89 antagonist, such as those described herein, may have. The human ZSIG89 cDNA can be used to isolate murine ZSIG89 mRNA, cDNA and genomic DNA, which are subsequently used to generate knockout mice. These mice may be employed to study the ZSIG89 gene and the protein encoded thereby in an in vivo system, and can be used as in vivo models for corresponding human diseases. Moreover, transgenic mice expression of ZSIG89antisense polynucleotides or ribozymes directed against ZSIG89, described herein, can be used analogously to transgenic mice described above.

ZSIG89 polypeptides, variants, and fragments thereof, may be useful as replacement therapy for disorders associated with cell-cell interactions, including disorders related to, for example, immunology, and epithelial disorders, in general.

A less widely appreciated determinant of tissue morphogenesis is the process of cell rearrangement: Both cell motility and cell-cell adhesion are likely to play central roles in morphogenetic cell rearrangements. Cells need to be able to rapidly break and probably simultaneously remake contacts with neighboring cells. See Gumbiner, B. M., Cell 69:385-387, 1992. As a secreted protein in tissues of the tissues lining the respiratory and gastrointestinal tracts, including lung and oral tissues, ZSIG89 can play a role in intercellular rearrangement in these and other tissues.

ZSIG89 gene may be useful to as a probe to identify humans who have a defective ZSIG89 gene. The strong expression of ZSIG89 in tissues of tissues lining the respiratory and gastrointestinal tracts, including lung and oral tissues suggests that ZSIG89 polynucleotides or polypeptides can be used as measured as an indication of aberrant growth in these tissues. Thus, polynucleotides and polypeptides of ZSIG89, and mutations to them, can be used a diagnostic indicators of cancer in these tissues.

The polypeptides of the present invention are useful in studying cell adhesion and the role thereof in metastasis and may be useful in preventing metastasis, in particular metastasis in tumors of the tissues lining the respiratory and gastrointestinal tracts, including lung and oral tissues. Similarly, polynucleotides and polypeptides of ZSIG89 may be used to replace their defective counterparts in tumor or malignant tissues.

Cancer can be considered a development-related disease in that its etiology involves either the dedifferentiation of cells or the loss of growth control or replicative control in a cell. Many genes known to be tumor promoting or tumor suppressing also play roles during normal development. Examples of genes involved in development as well as neoplastic progression are the retinoblastoma gene (for review see DiCionmmo D., et al., Semin. Cancer Biol. 10(4):255-69, 2000) and the breast cancer susceptibility loci, BRCA1 and BRCA2 (See, for example, Chodosh, L. A., J. Mammary Gland Biol. Neoplasia. 3(4): 289-402, 1998). Since Zsig89 is transcribed in tumor cells as well as specific fetal tissues it may not only be a tumor marker but also directly involved in particular tumorigenic processes.

The activity and effect of ZSIG89 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Tumor models include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing ZSIG89, before implantation so that the protein is synthesized at the tumor site or intracellularly, rather than systemically. The mice normally develop visible tumors within days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., ZSIG89, on the ability of the tumor to recruit vasculature and undergo metastasis can thus be assessed. In addition, aside from using adenovirus, the implanted cells can be transiently transfected with ZSIG89. Moreover, purified ZSIG89 or ZSIG89-conditioned media can be directly injected in to this mouse model, and hence be used in this system. Use of stable ZSIG89 transfectants as well as use of induceable promoters to activate ZSIG89 expression in vivo are known in the art and can be used in this system to assess ZSIG89 induction of metastasis. For general reference see, O'Reilly M S, et al. Cell 79:315-328, 1994; and Rusciano D, et al. Murine Models of Liver Metastasis. Invasion Metastasis 14:349-361, 1995.

As a polypeptide in tissues of the lining the respiratory and gastrointestinal tracts, including lung and oral tissues, ZSIG89 polypeptide pharmaceutical compositions of the present invention may be useful in prevention or treatment of disorders associated with pathological regulation or the expansion of these tissues.

The polynucleotides of the present invention may also be used in conjunction with a regulatable promoter, thus allowing the dosage of delivered protein to be regulated.

The ZSIG89 polynucleotides of SEQ ID NO:1 map to chromosome 6 band p21.3. Thus, the present invention also provides reagents which will find use in diagnostic applications. For example, the ZSIG89 gene, a probe comprising ZSIG89 DNA or RNA or a subsequence thereof can be used to determine if the ZSIG89 gene is present on chromosome 6 band p21.3 or if a mutation has occurred. Detectable chromosomal aberrations at the ZSIG89 gene locus include, but are not limited to, aneuploidy, gene copy number changes, insertions, deletions, restriction site changes and rearrangements. These aberrations can occur within the coding sequence, within introns, or within flanking sequences, including upstream promoter and regulatory regions, and may be manifested as physical alterations within a coding sequence or changes in gene expression level.

Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995). Additional detectable aberrations include single nucleotide polymorphisms (SNPs), and sequence-tagged sites (STS's), which can be useful in paternity and/or forensic testing (Nickerson, D., et al., Proc. Natl. Acad. Sci. 87:8923-7, 1990).

In general, these diagnostic methods comprise the steps of (a) obtaining a genetic sample from a patient; (b) incubating the genetic sample with a polynucleotide probe or primer as disclosed above, under conditions wherein the polynucleotide will hybridize to complementary polynucleotide sequence, to produce a first reaction product; and (iii) comparing the first reaction product to a control reaction product. A difference between the first reaction product and the control reaction product is indicative of a genetic abnormality in the patient. Genetic samples for use within the present invention include genomic DNA, cDNA, and RNA. The polynucleotide probe or primer can be RNA or DNA, and will comprise a portion of SEQ ID NOs:1 or 3, the complement of SEQ ID NOs:1 or 3, or an RNA equivalent thereof. Suitable assay methods in this regard include molecular genetic techniques known to those in the art, such as restriction fragment length polymorphism (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; Marian, Chest 108:255-65, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient's genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-8, 1991).

It has been shown that some HLA haplotypes may predispose humans to certain autoimmune diseases such as multiple sclerosis (Nickerson, et al., ibid.), inflammatory bowel disease, ulcerative colitis, and psoriasis, for example. As a protein that demonstrates expression in epithelial and endothelial tissues, ZSIG89 may be instrumental in sensory perception in these tissues. For example, ZSIG89 may be necessary for pain, pressure and/or temperature perception in epidermal and/or mucosal linings. Additionally, as a protein that is immune related, molecules of the present invention may be involved in the body's immune response to surface irritants, i.e., causing a rash or contact dermatitis, or hypersensitivity, for example. Thus, ZSIG89 polynucleotides, polypeptides or fragments thereof, as well as agonists and antagonists, may be useful in treating these diseases and symptoms.

The polypeptides of ZSIG89 may represent an antigenic marker for carcinomas, as well as sqaumous cell carcinoma of the lung, respiratory and gastrointestinal tracts, including tumors of the kidney, liver, and rectum. Thus, these polypeptides, or fragments thereof may be useful as antigens to produce humanized antibodies for treatment of these specific tumors. Additionally, these polypeptides and polypeptide fragments can be useful to generate vaccines for use cancer therapy.

For pharmaceutical use, the proteins of the present invention can be administered intravaginally, orally, rectally, parenterally (particularly intravenous or subcutaneous), intracistemally, intraperitoneally, topically (as douches, sprays, powders, ointments, drops or transdermal patch) bucally, or as a pulmonary or nasal inhalant. Intravenous administration will be by bolus injection or infusion over a typical period of one to several hours. In general, pharmaceutical formulations will include a ZSIG89 protein, alone, or in conjunction with a dimeric partner, in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of ZSIG89 is an amount sufficient to produce a clinically significant change in, tumor suppression, infection, myogenesis, inflammation, and infection in tissues lining the respiratory and gastrointestinal tracts, including lung and oral tissues. Similarly, a therapeutically effective amount of ZSIG89 is an amount sufficient to produce a clinically significant change in disorders associated with tissues lining the respiratory and gastrointestinal tracts, including lung and oral tissues.

The invention is further illustrated by the following non-limiting examples.

### EXAMPLES

### Example 1

### Tissue Distribution of Human ZSIG89 in Tissue Panels using PCR

A panel of cDNA samples from human tissues was screened for ZSIG89 expression using PCR. The panel was made in-house and contained 94 cDNA samples from marathon cDNA and cDNA samples from various normal and cancerous human tissues and cell lines as shown in Table 4, below. The cDNA samples came from in-house libraries or marathon cDNA preparations of RNA that were prepared in-house, or from a commercial supplier such as Clontech (Palo Alto, Calif.) or Invitrogen (Carlsbad, Calif.). The marathon cDNAs were made using the Marathon-Ready™ Kit (Clontech, Palo Alto, Calif.) and standardized to ensure an equal amount of cDNA was placed into each well. To assure quality of the panel samples, three tests for quality control (QC) were run: (1) To assess the RNA quality used for the libraries, the in-house cDNAs were tested for average insert size by PCR with vector oligos that were specific for the vector sequences for an individual cDNA library; (2) Standardization of the concentration of the cDNA in panel samples was achieved using standard PCR methods to amplify full length alpha tubulin or G3PDH cDNA; and (3) a sample was sent to sequencing to check for possible ribosomal or mitochondrial DNA contamination. The panel was set up in a 96-well format that included a human genomic DNA (Clontech, Palo Alto, Calif.) positive control sample. Each well contained approximately 0.2-100 pg/μl of cDNA. The PCR reactions were set up using oligos ZC28251 (SEQ ID NO:4) and ZC28252 (SEQ ID NO:5), TaKaRa Ex Taq™ (TAKARA Shuzo Co LTD, Biomedicals Group, Japan), and Rediload dye (Research Genetics, Inc., Huntsville, Ala.). The amplification was carried out as follows: 1 cycle at 94° C. for 2 minutes, 35 cycles of 94° C. for 30 seconds, 62.8° C. for 30 seconds and 72° C. for 30 seconds, followed by 1 cycle at 72° C. for 5 minutes. About 10 μl of the PCR reaction product was subjected to standard Agarose gel electrophoresis using a 2% agarose gel. The correct predicted DNA fragment size was observed in fetal liver, fetal skin, spinal cord, trachea, lung tumor, rectal tumor, and genomic. Faint signals were also seen in a human prostate epithelium cell line that had been transformed with human papillomavirus, adrenal gland, prostate smooth muscle cell line, CD3+, fetal brain, pituitary, salivary gland, testis, and HepG2 (pancreas and liver library).

The DNA fragment from genomic, lung tumor, testis and fetal brain were excised and purified using a Gel Extraction Kit (Qiagen, Chatsworth, Calif.) according to manufacturer's instructions, and sent to sequencing. Sequence from genomic and lung tumor confirmed fragment to be ZSIG89.

TABLE 4

| Tissue/Cell line | # tested |
|---|---|
| Adrenal gland | 1 |
| Bladder | 1 |
| Bone Marrow | 1 |
| Brain | 1 |
| Cervix | 1 |
| Colon | 1 |
| Fetal brain | 1 |
| Fetal heart | 1 |
| Fetal kidney | 1 |
| Fetal liver | 1 |
| Fetal lung | 1 |
| Fetal muscle | 1 |
| Fetal skin | 1 |
| Heart | 2 |
| K562 (ATCC # CCL-243) | 1 |
| Kidney | 1 |
| Liver | 1 |
| Lung | 1 |
| Lymph node | 1 |
| Melanoma | 1 |
| Pancreas | 1 |
| Pituitary | 1 |
| Placenta | 1 |
| Prostate | 1 |
| Rectum | 1 |
| Salivary Gland | 1 |
| Skeletal muscle | 1 |
| Small intestine | 1 |
| Spinal cord | 1 |
| Spleen | 1 |
| Stomach | 1 |
| Testis | 2 |
| Thyroid | 1 |
| Uterus | 1 |
| Gastric tumor | 1 |
| Liver tumor | 1 |
| Ovarian tumor | 1 |
| Uterus tumor | 1 |
| Bone marrow | 3 |
| Fetal brain | 3 |
| Islet | 2 |
| Prostate | 3 |
| RPMI#1788 (ATCC # CCL-156) | 2 |
| Testis | 4 |
| Thyroid | 2 |
| W138 (ATCC # CCL-75 | 2 |
| ARIP (ATCC # CRL-1674 - rat) | 1 |
| HaCat - human keratinocytes | 1 |
| HPV (ATCC # CRL-2221) | 1 |
| Adrenal gland | 1 |
| Prostate SM | 2 |
| CD3+ selected PBMC's (stimulated) | 1 |
| HPVS | 1 |
| (ATCC # CRL-2221) - selected | |
| Heart | 1 |
| Pituitary | 1 |
| Placenta | 2 |
| Salivary gland | 1 |
| HL6O (ATCC # CCL-240) | 3 |
| Platelet | 1 |
| HBL-100 | 1 |
| Renal mesangial | 1 |
| T-cell | 1 |
| Neutrophil | 1 |
| MPC | 1 |
| Hut-102 (ATCC # TIB-162) | 1 |
| Endothelial | 1 |
| HepG2 (ATCC # HB-8065) | 1 |
| Fibroblast | 1 |
| E. Histo | 1 |
| Thymus | 1 |
| Trachea | 1 |
| Esophagus tumor | 1 |
| Kidney tumor | 1 |
| Lung tumor | 1 |
| Rectal tumor | 1 |

### Example 2

### Construct for Generating Human ZSIG89 Transgenic Mice

Oligonucleotides were designed to generate a PCR fragment containing a consensus Kozak sequence and the exact human ZSIG89 coding region. These oligonucleotides were designed with an FseI site at the 5′ end and an AscI site at the 3′ end to facilitate cloning into pTgl2-8 MT.

PCR reactions were carried out using Advantage® cDNA polymerase (Clontech) to amplify a human ZSIG89 cDNA fragment. About 200 ng of human ZSIG89 polynucleotide template (Example 1), and oligonucleotides ZC27355 (SEQ ID NO:6) and ZC27356 (SEQ ID NO:7) were used in the PCR reaction. PCR reaction conditions were as follows: 95° C. for 5 minutes; cycles of 95° C. for 60 seconds, 61° C. for 60 seconds, and 72° C. for 90 seconds; and 72° C. for 7 minutes; followed by a 4° C. hold. PCR products were separated by agarose gel electrophoresis and purified using a QiaQuick™ (Qiagen) gel extraction kit. The isolated, approximately 980bp, DNA fragment was digested with FseI and AscI (New England BioLabs), ethanol precipitated and ligated into pTg12-8 MT that was previously digested with FseI and AscI. The pTg12-8 MT plasmid, designed for expression of a gene of interest in transgenic mice, contains an expression cassette flanked by 10 kb of MT-15′ DNA and 7 kb of MT-13′ DNA. The expression cassette is comprised of the MT-1 promoter, the rat insulin II intron, a polylinker for the insertion of the desired clone, and the human growth hormone poly A sequence.

About one microliter of the ligation reaction was electroporated into DH10B ElectroMax® competent cells (GIBCO BRL, Gaithersburg, Md.) according to manufacturer's direction and plated onto LB plates containing 100 μg/ml ampicillin, and incubated overnight. Colonies were picked and grown in LB media containing 100 μg/ml ampicillin. Miniprep DNA was prepared from the picked clones and screened for the ZSIG89 insert by restriction digestion with EcoRI and subsequent agarose gel electrophoresis and analysis. Maxipreps of the correct pTg12-8 MT ZSIG89 construct, as verified by sequence analysis, were performed. A SalI fragment containing the 5′ and 3′ flanking sequences, the MT promoter, the rat insulin II intron, ZSIG89 cDNA and the human growth hormone poly A sequence was prepared and used for microinjection into fertilized murine oocytes.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide comprising residues 27 to 325 of SEQ ID NO:2.

2. The isolated polypeptide of according to claim 1 wherein the polypeptide comprises residues 1 to 325 of SEQ ID NO:2.

3. An isolated polynucleotide encoding the polypeptide according to claim 1.

4. The isolated polynucleotide according to claim 3, wherein the polypeptide molecule comprises residues 1 to 325 of SEQ ID NO:2.

5. The isolated polynucleotide according to claim 4, wherein the isolated polypeptide has an amino acid sequence that is at least 90% identical to the polypeptide sequence as shown in SEQ ID NO:2.

6. The isolated polynucleotide according to claim 5, wherein any difference between the isolated polypeptide and the polypeptide as shown in SEQ ID NO:2 is due to conservative amino acid substitution.

7. An expression vector comprising the following operably linked elements:
a) a transcription promoter;
b) a DNA segment wherein the DNA segment is a polynucleotide encoding the polypeptide of claim 1; and
c) a transcription terminator.

8. The expression vector according to claim 7 wherein the DNA segment contains an affinity tag.

9. A cultured cell into which has been introduced the expression vector according to claim 7, wherein said cell expresses the polypeptide encoded by the DNA segment.

10. A method of producing a polypeptide comprising culturing a cell according to claim 9, whereby said cell expresses the polypeptide encoded by the DNA segment; and recovering the polypeptide.

11. The method of claim 10, comprising isolating the ZSIG89 protein from the cultured cell.

12. The polypeptide produced by the method of claim 10.

13. A method of producing an antibody comprising the following steps in order:
inoculating an animal with a polypeptide selected from the group consisting of:
d) a polypeptide comprising residues 1 to 26 of SEQ ID NO:2;
e) a polypeptide comprising residues 27 to 325 of SEQ ID NO:2; and
f) a polypeptide comprising residues 1 to 325 of SEQ ID NO:2.
wherein the polypeptide elicits an immune response in the animal to produce the antibody; and isolating the antibody from the animal.
(a) An antibody produced by the method of claim 13.
(b) The antibody according to claim 14, wherein the antibody specifically binds to a residues 1 to 325 of SEQ ID NO:2.

16. An epitope-bearing portion of the polypeptide as shown in SEQ ID NO:2, wherein the epitope bearing portion comprises 30 contiguous amino acids.

17. A method of producing an antibody comprising the following steps in order:
inoculating an animal with the epitope-bearing portion according to claim 16; wherein the polypeptide elicits an immune response in the animal;
producing the antibody in the animal; and
isolating the antibody from the animal,
wherein the antibody binds to the epitope-bearing portion.

18. An antibody produced by the method of claim 17.

19. The antibody of claim 18, wherein the antibody specifically binds to residues 1 to 325 of SEQ ID NO:2.

20. An antibody that binds to the polypeptide of claim 2, wherein the antibody is a monoclonal antibody.

21. A method of detecting the presence of ZSIG89 gene expression in a genetic sample, comprising:
(a) obtaining the genetic sample;
(b) incubating the genetic sample with a polynucleotide probe or primer, wherein the polynucleotide probe or primer comprises a portion of the polynucleotide according to claim 3, under conditions wherein the polynucleotide will hybridize to a complementary polynucleotide sequence;
(c) producing a reaction product; and
(d) detecting the formation of hybrids of the polynucleotide probe or primer and the genetic sample in the reaction product,
wherein the presence of the hybrids indicates the presence of ZSIG89 gene expression in the genetic sample.

22. A method of detecting the presence of ZSIG89 gene expression in a biological sample, comprising:
(a) obtaining the biological sample;
(b) contacting the biological sample with an antibody or antibody fragment under conditions wherein the contacting allows the binding of the antibody or antibody fragment to the biological sample, wherein the antibody or antibody fragment specifically binds with the polypeptide according to claim 1; and
(c) detecting the presence bound antibody or antibody fragment, wherein the presence of the bound antibody or antibody fragment indicates the presence of ZSIG89 gene expression.
